(19) United States
(12) Patent Application Publication
(10) Pub. No.: US 2024/0225869 A1
(43) Pub. Date: Jul. 11, 2024

(54) APPARATUSES AND METHODS OF HYBRID STENT PROSTHESES

(71) Applicant: Merit Medical Systems, Inc., South Jordan, UT (US)

(72) Inventors: John Hall, Bountiful, UT (US); Wayne Mower, Bountiful, UT (US); Christopher Cindrich, Highland, UT (US)

(21) Appl. No.: 18/404,447

(22) Filed: Jan. 4, 2024

Related U.S. Application Data
(60) Provisional application No. 63/478,598, filed on Jan. 5, 2023.

(51) Int. Cl. A61F 2/954; A61F 2/958; A61F 2/966

(52) U.S. Cl. CPC A61F 2/954; A61F 2/958; A61F 2/966

(57) ABSTRACT

Hybrid stent prosthesis and related methods for their use are described herein. A hybrid stent prosthesis may include a self-expanding stent frame, a balloon-expandable stent frame, and a cover integrating the self-expanding stent frame and the balloon-expandable stent frame. In some cases, the hybrid stent prosthesis may further include a transition zone and/or one or more markers. In some circumstances, the hybrid stent prosthesis may be advanced through the anatomy of a patient by a delivery catheter system and deployed at a deployment site by a deployment system of the delivery catheter system. A balloon inflation corresponding to the balloon-expandable stent frame may anchor the hybrid stent prosthesis. After anchoring, the self-expanding stent frame may be allowed to expand to its deployed configuration. The distal end of the balloon-expandable stent frame may then be further expanded to match anatomy. In cases, the hybrid stent prosthesis deploys with/in a parent prosthesis.

### CROSS-REFERENCE TO RELATED APPLICATIONS

This application claims priority to U.S. Provisional Patent Application No. 63/478,598 filed on Jan. 5, 2023, the disclosure of which is incorporated herein, in its entirety, by this reference.

### TECHNICAL FIELD

The present application relates to stent prostheses and methods of using the same.

### BRIEF DESCRIPTION OF THE SEVERAL VIEWS OF THE DRAWINGS

FIG. 1 illustrates a hybrid stent prosthesis, according to embodiments herein.

FIG. 2 illustrates a hybrid stent prosthesis, according to embodiments herein.

FIG. 3 illustrates a hybrid stent prosthesis, according to embodiments herein.

FIG. 4 illustrates a hybrid stent prosthesis, according to embodiments herein.

FIG. 5 illustrates a hybrid stent prosthesis, according to embodiments herein.

FIG. 6 illustrates a deployment system for a hybrid stent prosthesis, according to embodiments herein.

FIG. 7A through FIG. 7D illustrate together the use of a fenestrated endovascular prosthesis with a hybrid stent prosthesis, according to embodiments herein.

FIG. 8 illustrates a method of deploying a hybrid stent prosthesis, according to embodiments herein.

### DETAILED DESCRIPTION

Delivery catheter systems may be configured to deliver one or more medical appliances or systems to a location within a patient's body and deploy the medical appliance or system within the patient's body. For example, such a delivery catheter system may be configured to be advanced from an insertion site at the outside of an anatomical system to a treatment location within the anatomical system. For example, a delivery catheter system may be configured to be advanced through bends, turns, or other structures within the anatomy of the vasculature.

A stent prosthesis may be disposed within the delivery catheter system as part of a deployment system of the delivery catheter system such that a practitioner may deploy the stent prosthesis from a distal end of the delivery catheter system through manipulation of one or more components of, for example, a handle assembly of the delivery catheter system.

Stent prostheses may be deployed in various body lumens for a variety of purposes. Stent prostheses may be deployed, for example, in the arterial system for a variety of therapeutic purposes including the treatment of occlusions within the lumens of that anatomical system. It will be appreciated that the current disclosure may be applicable to stent prostheses designed for the central venous system, peripheral vascular system, abdominal aortic aneurism system, bronchial system, esophageal system, biliary system, or any other system of the human body. Further, the present disclosure may equally be applicable to other prosthesis such as grafts.

Accordingly, it will be understood that while specific examples recited herein may refer to deployment of cardiovascular stent prostheses within a cardiovascular system, analogous concepts and devices may be used in/with various other anatomical systems of the body, including for placement and deployment of medical appliances in the gastrointestinal tract (including, for example, within the esophagus, intestines, stomach, small bowel, colon, and biliary duct); the respiratory system (including, for example, within the trachea, bronchial tubes, lungs, nasal passages, and sinuses); or any other location within the body, both within bodily lumens (for example, the ureter, the urethra, etc.) and within other bodily structures.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “proximal” and “distal” are used herein to refer to opposite locations relative to a medical device in use by a practitioner. The proximal end of the device is defined as the end of the device closest to the practitioner when the device is in use by the practitioner. The distal end is the end opposite the proximal end.

Embodiments may be understood by reference to the drawings, wherein like parts are designated by like numerals throughout. It will be understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

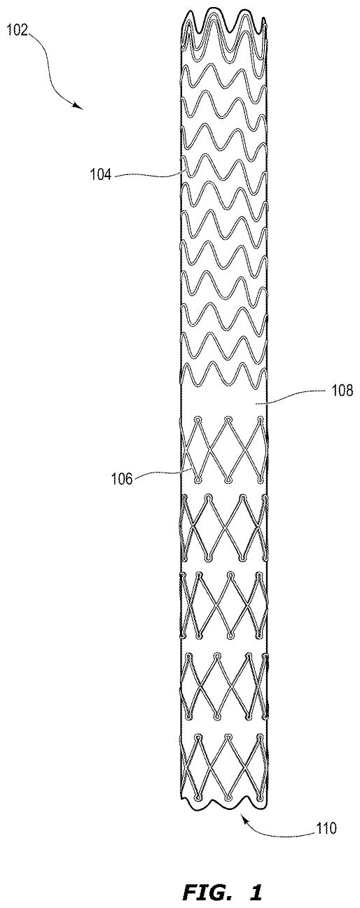

FIG. 1 illustrates a hybrid stent prosthesis 102, according to embodiments herein. The hybrid stent prosthesis 102 of FIG. 1 is illustrated in a deployed (expanded) configuration.

The hybrid stent prosthesis 102 includes a self-expanding stent frame 104, a balloon-expandable stent frame 106, and a cover 108 that integrates each of the self-expanding stent frame 104 and the balloon-expandable stent frame 106. Note that the hybrid stent prosthesis 102 is not limited to the illustrated embodiment of FIG. 1. While the hybrid stent prosthesis 102 illustrated in FIG. 1 includes a single self-expanding stent frame 104 and a single balloon-expandable stent frame 106, other embodiments of the hybrid stent prosthesis 102 may include multiple self-expanding stent frames 104 and/or multiple balloon-expandable stent frames 106. For example, the hybrid stent prosthesis 102 may include the self-expanding stent frame 104, the balloon-expandable stent frame 106, and an additional self-expanding stent frame. The cover 108 may integrate the additional self-expanding stent frame with the balloon-expandable stent frame 106 positioned between the self-expanding stent frame 104 and the additional self-expanding stent frame. In some embodiments, the hybrid stent prosthesis 102 may include the self-expanding stent frame 104, the balloon-expandable stent frame 106, and an additional balloon-expandable stent frame. The cover 108 may integrate the additional balloon-expandable stent frame with the self-expanding stent frame 104 positioned between the balloon-expandable stent frame 106 and the additional balloon-expandable stent frame.

The self-expanding stent frame 104 is configured such that it automatically expands to a deployed configuration (e.g., as illustrated) when it is not otherwise radially constrained. For example, the self-expanding stent frame 104 may be helical in nature, and may be made of a self-expanding and/or super elastic material that has been treated such that it tends to return to a given shape when it is not otherwise constrained when in the human body. For example, a self-expanding stent frame 104 may be made of Nitinol that has (previously) been set in a desired shape (e.g., the illustrated helical configuration of FIG. 1) and heated to set that shape, such that it returns to that set shape once it is otherwise unconstrained and at a typical human body temperature.

Due to the self-expanding nature of the self-expanding stent frame 104, the self-expanding stent frame 104 may be constrained during delivery of the hybrid stent prosthesis 102 to a deployment site. In examples herein, a catheter sleeve may constrain a self-expanding stent frame 104 in a collapsed configuration against an elongate tubular member of a delivery catheter system during such delivery.

Note that the self-expanding stent frame 104 is not limited to the illustrated embodiment of FIG. 1 (using a helical pattern). A self-expanding stent frame 104 as contemplated could comprise one or more of a variety of possible different patterns, such as a diamond pattern, a zig-zag pattern, a wave pattern, or any other suitable pattern.

The balloon-expandable stent frame 106 is configured to be expandable to a deployed position (e.g., as illustrated) from an unexpanded/undeployed configuration via the inflation of one or more balloons from within the portion of the hybrid stent prosthesis 102 corresponding to the balloon-expandable stent frame 106 that exert an outward radial force on the balloon-expandable stent frame 106. The balloon-expandable stent frame 106 may be formed of any suitable material, such as nickel-titanium alloy, stainless steel, cobalt-chromium, platinum, polymers, etc. The material, pattern, and wire diameter of the balloon-expandable stent frame 106, or the wall thickness and strut width of the balloon-expandable stent frame 106, may be configured to provide a chronic radial outwardly directed force and a resistance to an inward radial compression when disposed in a body lumen of a patient. The balloon-expandable stent frame 106 may be configured to provide a radial stiffness and radial strength to resist a local or inwardly compressive force when deployed in a body lumen of a patient.

In the embodiment of FIG. 1, the balloon-expandable stent frame 106 has been expanded/deployed to its nominal diameter corresponding to an expanded configuration. However, it will be understood that balloon-expandable stent frame 106 is configurable such that the diameter of different portions of the balloon-expandable stent frame 106 may be selectively made different when in a deployed configuration. Specifically, in some situations and in some embodiments, the balloon-expandable stent frame 106 may be serially dilated so that the balloon-expandable stent frame 106 and/or a portion thereof expands past a nominal diameter of the balloon-expandable stent frame 106. This may be useful in the treatment of the implant site due diseased, misshapen, and/or damaged vessels, and/or to more properly fit vessels having variable diameters. Serial dilation of the balloon-expandable stent frame 106 may be accomplished through the use of gradually larger diameter balloons to enable the balloon-expandable stent frame 106 to expand past its nominal diameter.

Note that the balloon-expandable stent frame 106 is not limited to the illustrated embodiment of FIG. 1 (using a diamond pattern). A balloon-expandable stent frame 106 as contemplated could comprise one or more of a variety of possible different patterns, such as a diamond pattern, a zig-zag pattern, a wave pattern, or any other suitable pattern.

The cover 108 may be formed of a variety of materials and/or layers of materials, including biocompatible materials that are resistant to passage of fluid through the cover 108. For example, cover 108 may be formed of polyethylene terephthalate, polyurethane, silicone rubber, nylon, fluoropolymer, polyester, etc. A thickness of the cover 108 may range from about 0.025 mm to about 0.5 mm.

In certain embodiments, the cover 108 may be impermeable to tissue cell ingrowth into and/or tissue cell migration through the cover 108, for example, to prevent or discourage stenosis of the cover 108. Additionally or alternatively, in some embodiments, the cover 108 can be impermeable to fluid such that fluid is prevented from leaking from the inside of the hybrid stent prosthesis 102 to the exterior of the hybrid stent prosthesis 102.

In some embodiments, the cover 108 may comprise a plurality of layers. In some embodiments, the cover 108 may comprise an outer layer, a tie layer, and an inner layer. In some embodiments the tie layer may be configured to promote bonding between the outer layer and the inner layer. In other embodiments the tie layer may further be configured to provide certain properties to the hybrid stent prosthesis 102 as a whole, such as stiffness or tensile strength. In some embodiments, the inner layer may be rotational spun polytetrafluoroethylene (PTFE) (which may resist fibrin deposition and platelet adhesion on the interior surfaces of the hybrid stent prosthesis 102), the tie layer may be fluorinated ethylene propylene (FEP), and the outer layer may be expanded PTFE (ePTFE). In some embodiments, the tie layer may be elastic material, a thermoplastic material, silicone, and the like. However, the present disclosure is not so limited, and other configurations of one or more layers of the cover 108 are also contemplated.

Additionally, in embodiments where both the inner layer and the outer layer are porous in nature, the tie layer may be configured to create an impermeable layer between the two porous layers. In such embodiments the hybrid stent prosthesis 102 may permit tissue ingrowth, tissue attachment, and/or healing on both the inner and outer surfaces of the cover 108 while still preventing tissue outside of the cover 108 from growing into the lumen of the hybrid stent prosthesis 102 and occluding the lumen. Thus, the tie layer may be configured to create a mid-layer portion of a construct and the tie layer is tissue or cell impermeable to inhibit tissue or cellular ingrowth into the layer or to be impervious to tissue migration into or through the layer or to substantially inhibit tissue migration.

The cover 108 may integrate the self-expanding stent frame 104 and the balloon-expandable stent frame 106 such that when the hybrid stent prosthesis is in a deployed configuration (e.g., when each of the self-expanding stent frame 104 and balloon-expandable stent frame 106 have been expanded), a lumen 110 of the hybrid stent prosthesis 102 passes through the self-expanding stent frame 104 and the balloon-expandable stent frame 106.

In the embodiment of FIG. 1, the portion of the cover 108 corresponding to the balloon-expandable stent frame 106 has been expanded/deployed to its nominal diameter, which may correspond to a nominal diameter of the balloon-expandable stent frame 106 as described herein. However, it will be understood that (at least) a portion of the cover 108 along the balloon-expandable stent frame 106 is configurable such that the diameter of cover 108 along the balloon-expandable stent frame 106 varies when in a deployed configuration. In other words, the diameter of the cover 108 along the balloon-expandable stent frame 106 may “follow” a correspondingly varying diameter of the balloon-expandable stent frame 106, as the case may be.

A functional elasticity of the cover 108 may allow for diameter variations without disabling the functionality of the portion of the cover 108 along the balloon-expandable stent frame 106 (as this functionality is described herein). For example, in some embodiments, the hybrid stent prosthesis 102 may be manufactured so that the cover 108 maintains its tissue impermeability up to a specific ratio of expansion of the balloon-expandable stent frame 106 beyond the nominal diameter for the cover 108. In some cases, this may be accomplished by manufacturing a tie layer of the cover 108 such that no tearing occurs in the tie layer attendant to this type of expansion. In some cases, the functional elasticity of the cover 108 may correspond to a maximum deployable diameter of the balloon-expandable stent frame 106.

In some embodiments, the cover 108 may maintain its tissue impermeability up to a fifty percent expansion of the balloon-expandable stent frame 106 beyond the nominal diameter of the cover 108. Embodiments wherein the cover 108 maintains its tissue impermeability up to a fifty percent expansion, forty percent expansion, thirty percent expansion, twenty percent expansion, or embodiments wherein the cover 108 maintains its tissue impermeability greater than twenty percent, greater than thirty percent, or greater than forty percent expansion of the balloon-expandable stent frame 106 beyond the nominal diameter of the cover 108 are all within the scope of this disclosure. In some embodiments, cover 108 maintains its tissue impermeability up to a 6 mm change in diameter relative to it nominal diameter.

In some embodiments, a luminal surface of the cover 108 (the inside surface of the cover 108 most directly defining the lumen 110) is understood to be continuous between the self-expanding stent frame 104 and the balloon-expandable stent frame 106. A continuous luminal surface may result by manufacturing the cover 108 as a single unit that integrates the self-expanding stent frame 104 and the balloon-expandable stent frame 106 as described. This may be compared to a hybrid stent prosthesis having a non-continuous luminal surface (where, for example, a cover is made up of separately manufactured cover pieces each separately integrating one of a self-expanding stent frame and a balloon-expandable stent frame 106 that are then sutured/overlapped/otherwise joined or connected together in a non-continuous manner). The continuous nature of the luminal surface of the cover 108 may promote flow through the hybrid stent prosthesis 102 better than alternative cases where a luminal surface of a cover is non-continuous (e.g., due to piece-wise manufacturing of the cover of the alternative case).

In some embodiments, the hybrid stent prosthesis 102, once deployed at a deployment site or deployment location, provides an appropriate channel for a desired flow of fluid at/through the deployment site. It may be that the region around deployment site is diseased, misshapen, and/or damaged, and that the deployment of the hybrid stent prosthesis 102 can correct and/or ameliorate attendant issues.

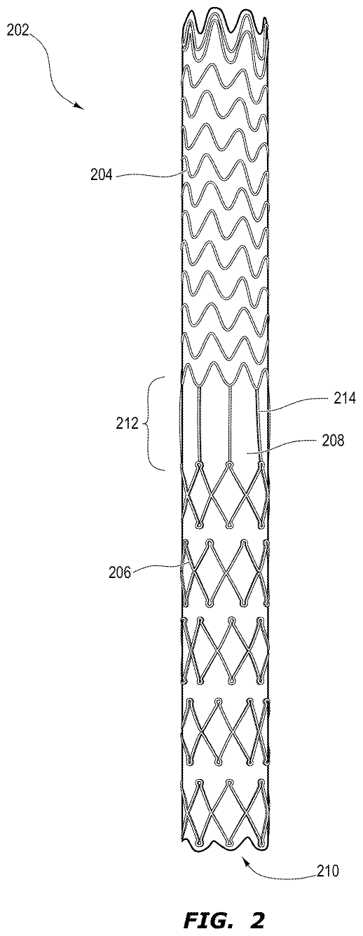

FIG. 2 illustrates a hybrid stent prosthesis 202, according to embodiments herein. The hybrid stent prosthesis 202 includes a self-expanding stent frame 204, a balloon-expandable stent frame 206, a cover 208, a lumen 210, and a transition zone 212.

The hybrid stent prosthesis 202 may resemble other hybrid stent prostheses discussed herein in certain respects. For example, the self-expanding stent frame 204, the balloon-expandable stent frame 206, the cover 208, and the lumen 210 may correspond to analogous ones of these elements discussed in relation to other embodiments herein.

In some embodiments of the hybrid stent prosthesis 202, the transition zone 212 of the cover 208 and regions of the cover 208 integrating the self-expanding stent frame 204 and the balloon-expandable stent frame 206 include a single, continuous material (e.g., the cover 208 is continuous over the self-expanding stent frame 204, the balloon-expandable stent frame 206, and the transition zone 212). In some embodiments of the hybrid stent prosthesis 202, a first layering of the cover 208 in the transition zone 212 is different than a second layering of the cover 208 at an end of one of the self-expanding stent frame 204 and the balloon-expandable stent frame 206 that is adjacent to the transition zone 212.

In the embodiment illustrated in FIG. 2, the transition zone 212 of the hybrid stent prosthesis 202 may provide a separation or spacing between the self-expanding stent frame 204 and the balloon-expandable stent frame 206. In some cases, it may be that an immediate adjacency of the self-expanding stent frame 204 with the balloon-expandable stent frame 206 within the hybrid stent prosthesis 202 is undesirable due to forces that the self-expanding stent frame 204 and the balloon-expandable stent frame 206 may exert upon each other through the cover 208.

For example, it may be that the adjacent ends of the self-expanding stent frame 204 and the balloon-expandable stent frame 206 have differing nominal diameters and/or are desired to be deployed at different diameters (e.g., through a sizing of the balloon-expandable stent frame 206 that is different to the nominal diameter of the self-expanding stent frame 204). In such a case, adjacent ends of the self-expanding stent frame 204 and the balloon-expandable stent frame 206, when relatively close to each other, may exert competing outward/inward radial forces on each other through the cover 208 when the hybrid stent prosthesis 202 is in the deployed configuration. This may cause an unintended/undesirable shape in the corresponding portion of the hybrid stent prosthesis 202 when in the deployed configuration.

As another example, it may be that, as part of a deployment procedure, one or the other of the self-expanding stent frame 204 and the balloon-expandable stent frame 206 is desired to take its respective expanded state prior to the expansion of the other of the self-expanding stent frame 204 and the balloon-expandable stent frame 206. In such cases, if adjacent ends of the self-expanding stent frame 204 and the balloon-expandable stent frame 206 are relatively close to each other, a first of these ends of the one of the self-expanding stent frame 204 and the balloon-expandable stent frame 206 that remains in its non-expanded configuration during this first deployment stage may interfere with (e.g., exert an inward radial force on) a second of these ends of the other of the self-expanding stent frame 204 and the balloon-expandable stent frame 206 that is being expanded during this first deployment stage, thereby ultimately interfering with the desired expansion during (at least) the first deployment stage.

Accordingly, to alleviate these issues, a transition zone 212 may be provided as part of the hybrid stent prosthesis 202. As illustrated, the transition zone 212 corresponds to a portion of the cover 208 of the hybrid stent prosthesis 202 that is between the self-expanding stent frame 204 and the balloon-expandable stent frame 206 (rather than directly supported by/integrated with either of the hybrid stent prosthesis 202 or the self-expanding stent frame 204). This transition zone 212 accordingly provides an amount of “slack” between the self-expanding stent frame 204 and the balloon-expandable stent frame 206 through the cover 208 that may reduce or eliminate the undesirable impacts of counter-forces through the cover 208 as between the self-expanding stent frame 204 and the balloon-expandable stent frame 206, as there are described herein.

Because the portion of the cover 208 corresponding to the transition zone 212 is not directly supported by either of the self-expanding stent frame 204 or the balloon-expandable stent frame 206, it may be desirable to provide extra structure for the cover 208 within the transition zone 212. Accordingly, as illustrated, the cover 208 along the transition zone 212 of FIG. 2 is provided with a plurality of longitudinal beadings 214 of a reinforcing material. These longitudinal beadings 214 may be provided on a surface of the cover 208 (e.g., corresponding to either the outside surface of the hybrid stent prosthesis 202 and/or the internal (luminal) surface of the hybrid stent prosthesis 202), and/or may be provided internal to the cover 208.

These longitudinal beadings 214 may support/fortify the cover 208 within the transition zone 212. For example, the longitudinal beadings 214 may alleviate or prevent sagging through the transition zone 212, and/or may reinforce the overall structural integrity of the transition zone 212. The longitudinal beadings 214 may be made of, for example, FEP in some embodiments.

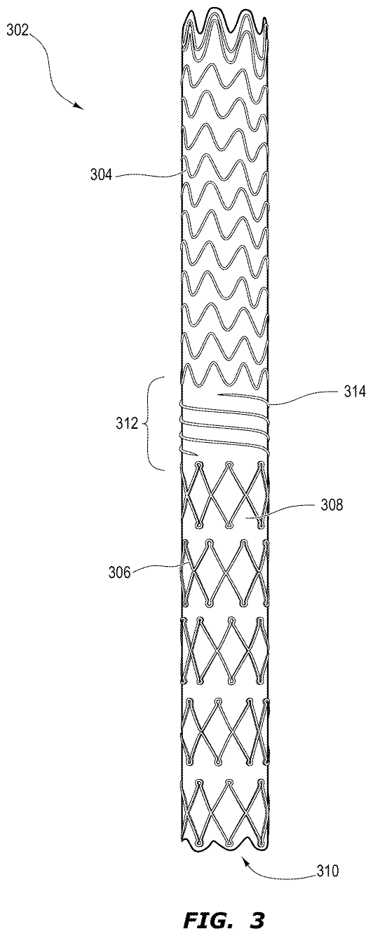

FIG. 3 illustrates a hybrid stent prosthesis 302, according to embodiments herein. The hybrid stent prosthesis 302 includes a self-expanding stent frame 304, a balloon-expandable stent frame 306, a cover 308, a lumen 310, and a transition zone 312.

The hybrid stent prosthesis 302 may resemble other hybrid stent prostheses discussed herein in certain respects. For example, the self-expanding stent frame 304, the balloon-expandable stent frame 306, the cover 308, the lumen 310 may correspond to analogous ones of these elements discussed in relation to other embodiments herein.

The hybrid stent prosthesis 302 may further include the transition zone 312. The transition zone 312 may be intended to have analogous functions and/or characteristics as other transition zones described herein.

Further, similarly to the portion of the cover 208 corresponding to the transition zone 212 of FIG. 2, the portion of the cover 308 corresponding to the transition zone 312 is not directly supported by either of the self-expanding stent frame 304 or the balloon-expandable stent frame 306, and it therefore may be desirable to provide extra structure for the cover 308 within the transition zone 312. Accordingly, as illustrated, the cover 308 along the transition zone 312 of FIG. 3 is provided with a plurality of helical beadings 314 of a reinforcing material. These helical beadings 314 may be provided on a surface of the cover 308 (e.g., corresponding to either the outside surface of the hybrid stent prosthesis 302 and/or the internal lumen surface of the hybrid stent prosthesis 302), and/or may be provided internal to the cover 308.

These helical beadings 314 may support/fortify the cover 308 within the transition zone 312. For example, the helical beadings 314 may alleviate or prevent sagging through the transition zone 312, and/or may reinforce the overall structural integrity of the transition zone 312. The helical beadings 314 may be made of, for example, FEP in some embodiments.

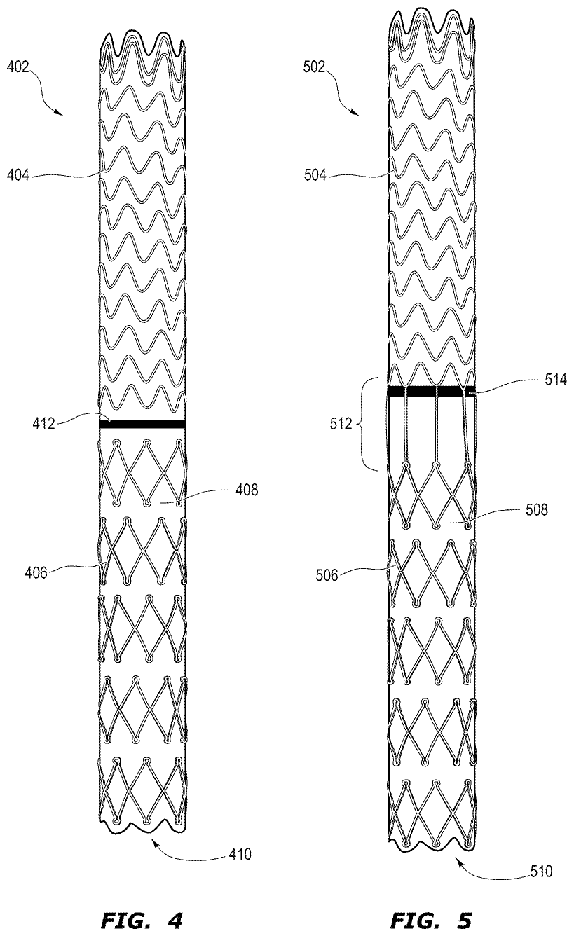

FIG. 4 illustrates a hybrid stent prosthesis 402, according to embodiments herein. The hybrid stent prosthesis 402 includes a self-expanding stent frame 404, a balloon-expandable stent frame 406, a cover 408, a lumen 410, and a marker 412.

The hybrid stent prosthesis 402 may resemble other hybrid stent prostheses discussed herein in certain respects. For example, the self-expanding stent frame 404, the balloon-expandable stent frame 406, the cover 408, and the lumen 410 may correspond to analogous ones of these elements discussed in relation to other embodiments herein.

In the embodiment illustrated in FIG. 4, a marker 412 is included in the hybrid stent prosthesis 402 (e.g., as part of the cover 408). The marker 412 can be formed from any suitable radiopaque material, such as gold, tantalum, or platinum-iridium alloy. Other materials are contemplated within the scope of this disclosure. The marker 412 may be used in conjunction with imaging systems employing radiography, fluoroscopy, and/or the like that can discernibility image the marker 412, thereby causing the marker 412 to act as an aid in the deployment of the hybrid stent prosthesis 402.

In the embodiment illustrated in FIG. 4, the marker 412 corresponds to the adjacent ends of the self-expanding stent frame 404 and the balloon-expandable stent frame 406. Accordingly, when deploying the hybrid stent prosthesis 402 under an imaging system, a practitioner may be aware of the position within the hybrid stent prosthesis 402 where the change from the self-expanding stent frame 404 to the balloon-expandable stent frame 406 occurs. This may aid in deployment and/or placement of the hybrid stent prosthesis 402.

In the embodiment shown in FIG. 4, the marker 412 has been illustrated as a radiopaque marker band that travels some or all of the diameter of the hybrid stent prosthesis 402 at the indicated location. However, this particular marker 412 has been provided by way of example and not by way of limitation. In other cases, the marker 412 may be any indicia at the provided location that may be provided on/within the hybrid stent prosthesis 402 and later discerned on/within the hybrid stent prosthesis 402 (e.g., via the use of an imaging system).

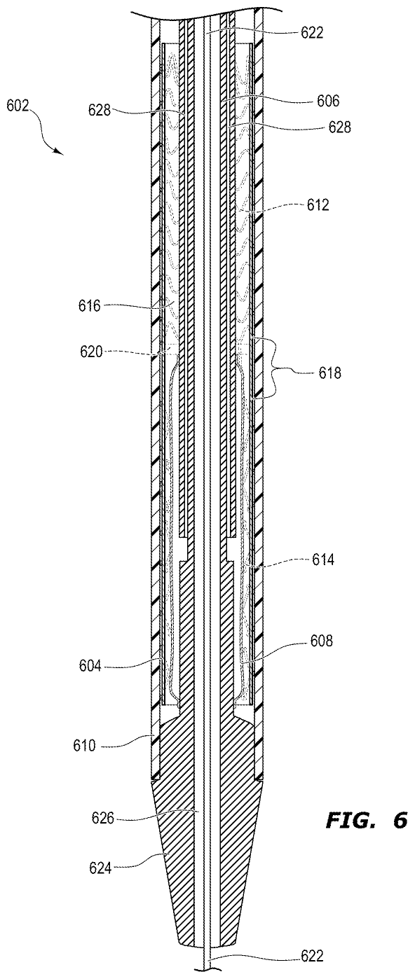

FIG. 5 illustrates a hybrid stent prosthesis 502, according to embodiments herein. The hybrid stent prosthesis 502 includes a self-expanding stent frame 504, a balloon-expandable stent frame 506, a cover 508, a lumen 510, a transition zone 512, and a marker 514.

The hybrid stent prosthesis 502 may resemble other hybrid stent prostheses discussed herein in certain respects. For example, the self-expanding stent frame 504, the balloon-expandable stent frame 506, the cover 508, the lumen 510, the transition zone 512, and the marker 514 may correspond to analogous ones of these elements discussed in relation to other embodiments herein.

In the embodiment illustrated in FIG. 5, the marker 514 is located close to the end of the self-expanding stent frame 504 that is adjacent to the transition zone 512 of the hybrid stent prosthesis 502. Accordingly, when deploying the hybrid stent prosthesis 502 under an imaging system, a practitioner may be aware of the position within the hybrid stent prosthesis 502 where the change from the transition zone 512 to the self-expanding stent frame 504 occurs. This may aid in deployment and/or placement of the hybrid stent prosthesis 502.

In the embodiment shown in FIG. 5, the marker 514 has been illustrated as a radiopaque marker band that travels some or all of the diameter of the hybrid stent prosthesis 502 at the indicated location. However, this particular marker 514 has been provided by way of example and not by way of limitation. In other cases, the marker 514 may be any indicia at the provided location that may be provided on/within the hybrid stent prosthesis 502 and later discerned on/within the hybrid stent prosthesis 502 (e.g., via the use of an imaging system).

It is noted that the number and/or placement of markers as described in embodiments herein is given by way of example and not by way of limitation. In other words, the existence of multiple markers in addition to/other than those shown in, for example, FIG. 4 and FIG. 5 is expressly contemplated as within the scope of this disclosure.

FIG. 6 illustrates a deployment system 602 for a hybrid stent prosthesis 604, according to embodiments herein. The deployment system 602 includes the hybrid stent prosthesis 604, an elongate tubular member 606, a balloon 608, and a catheter sleeve 610. The deployment system 602 may be understood to be part of/comprised in a delivery catheter system used by the practitioner to execute functions corresponding to the deployment system 602.

The hybrid stent prosthesis 604 resembles other hybrid stent prostheses discussed herein in certain respects. For example, the hybrid stent prosthesis 604 includes a self-expanding stent frame 612, a balloon-expandable stent frame 614, a cover 616, a transition zone 618, and a marker 620, each of which may correspond to analogous ones of these elements discussed in relation to other embodiments herein.

In FIG. 6, the hybrid stent prosthesis 604 is illustrated in a collapsed configuration around the elongate tubular member 606 of the deployment system 602. A reduced axial radius corresponding to the collapsed configuration of the hybrid stent prosthesis 604 may facilitate the delivery of the hybrid stent prosthesis 604 to a deployment site via the deployment system 602 through an anatomical system of a patient. As illustrated, the elongate tubular member 606 of the deployment system 602 may be configured with an internal lumen 626 that runs along a guidewire 622 that leads to a deployment site, such that the deployment system 602 arrives at the desired deployment site as it is pushed or otherwise navigated through the anatomical system of the patient. A tip 624 at the end of the elongate tubular member 606 may further facilitate this delivery through any tight spaces and/or sharp bends of the anatomical system.

The balloon 608 may be attached to and/or comprised in the elongate tubular member 606 and may be selectively inflatable by a practitioner controlling the deployment system 602. The balloon 608 may be in fluid communication with one or more lumens 628 of the elongate tubular member 606 that run back along the elongate tubular member 606 and out of the body of the patient, and through which fluid may be delivered and/or retrieved to and/or from the balloon to facilitate the selective inflation. The balloon 608 may be inflatable to a diameter that causes the balloon-expandable stent frame 614 of the hybrid stent prosthesis 604 that surrounds the balloon 608 to change from its collapsed configuration (as illustrated) to an expanded/deployed configuration.

The catheter sleeve 610 may hold all or part of the collapsed hybrid stent prosthesis 604 against the elongate tubular member 606 during delivery. In other words, during delivery, all or part of the hybrid stent prosthesis 604 may be “between” the elongate tubular member 606 and the catheter sleeve 610. This may aid in the delivery of the hybrid stent prosthesis 604 through the anatomical system of the patient, as this covering of the hybrid stent prosthesis 604 by the catheter sleeve 610 may prevent the covered portions of the hybrid stent prosthesis 604 from catching or snagging on anatomical features during travel of the deployment system 602 along the guidewire 622.

Further, the catheter sleeve 610 may be configured to constrain or restrict the self-expanding stent frame 612 against the elongate tubular member 606. In other words, the catheter sleeve 610 may be configured to constrain the self-expanding stent frame 612 to keep it in its collapsed configuration against the elongate tubular member 606 such that it is prevented from automatically expanding until the catheter sleeve 610 is retracted from or otherwise removed from along the self-expanding stent frame 612.

Note that while the embodiment illustrated in FIG. 6 shows that, during delivery of the deployment system 602 along the guidewire 622 to a deployment site, the catheter sleeve 610 is covering all portions of the hybrid stent prosthesis 604, it is contemplated that in some cases, the catheter sleeve 610 may instead cover only a portion of the hybrid stent prosthesis 604 during delivery (e.g., may cover only the portion of the hybrid stent prosthesis 604 corresponding to the self-expanding stent frame 612 and/or corresponding to the self-expanding stent frame 612 and the transition zone 618 during delivery).

Particular use cases and corresponding methods of delivery for a hybrid stent prosthesis are now discussed. It is noted that use cases and corresponding particulars of delivery methods for hybrid stent prostheses as these are expressly described herein are given by way of example and not by way of limitation. In other words, while disclosure herein may relate a use case and/or particulars of a delivery method for a hybrid stent prosthesis (e.g., in a particular anatomical context), such disclosure should be understood by way of example and not by way of limitation. Other anatomical contexts may apply and/or corresponding delivery method particulars for a hybrid stent prostheses could be used, depending on circumstance.

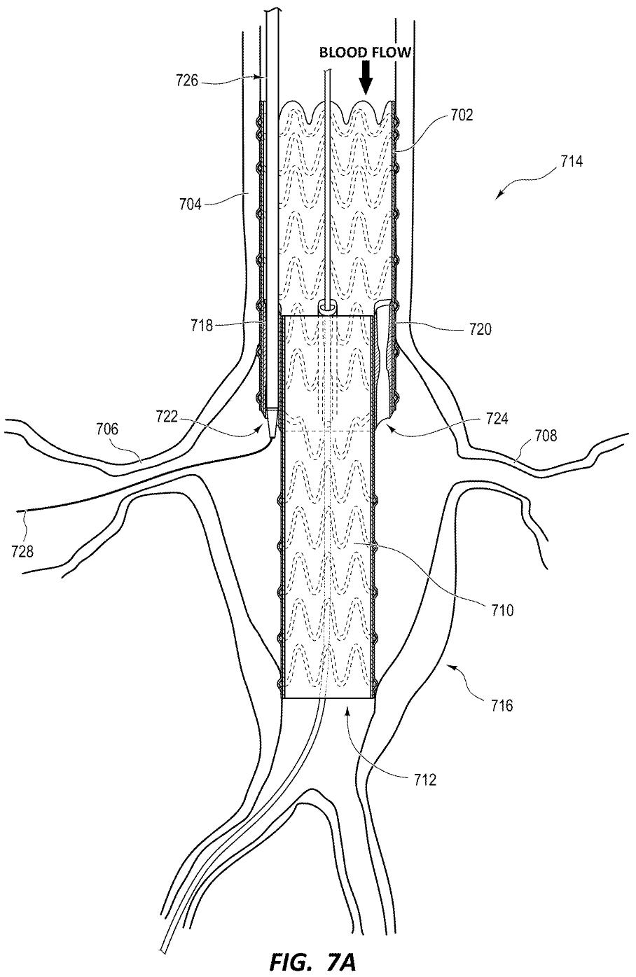

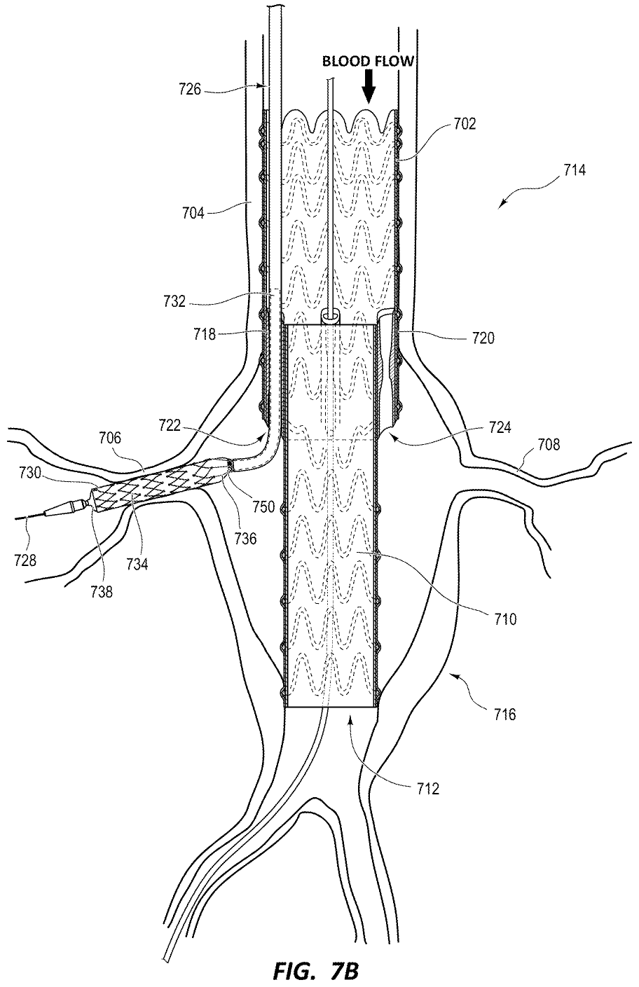

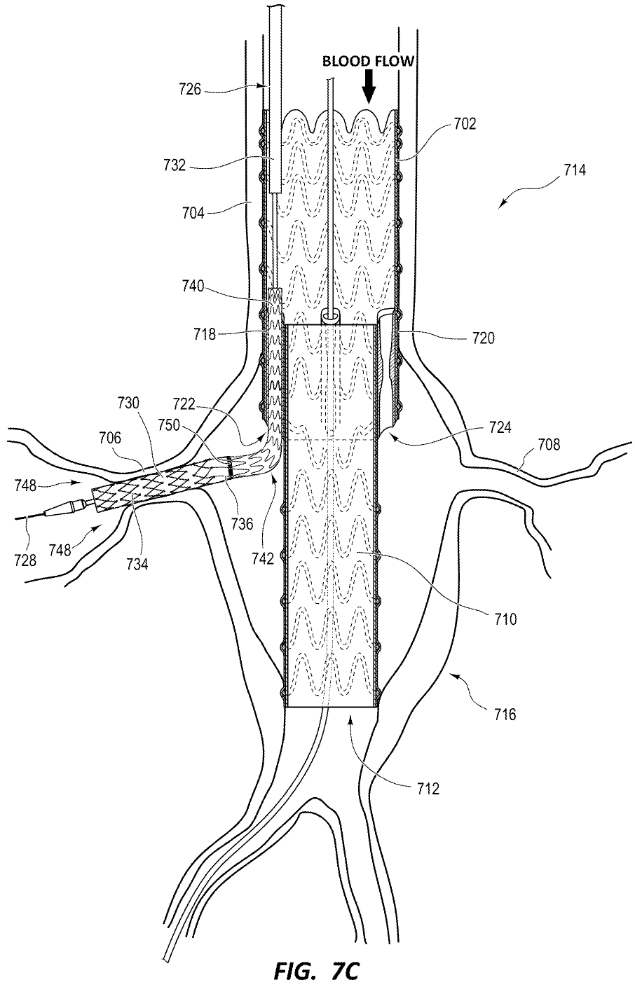

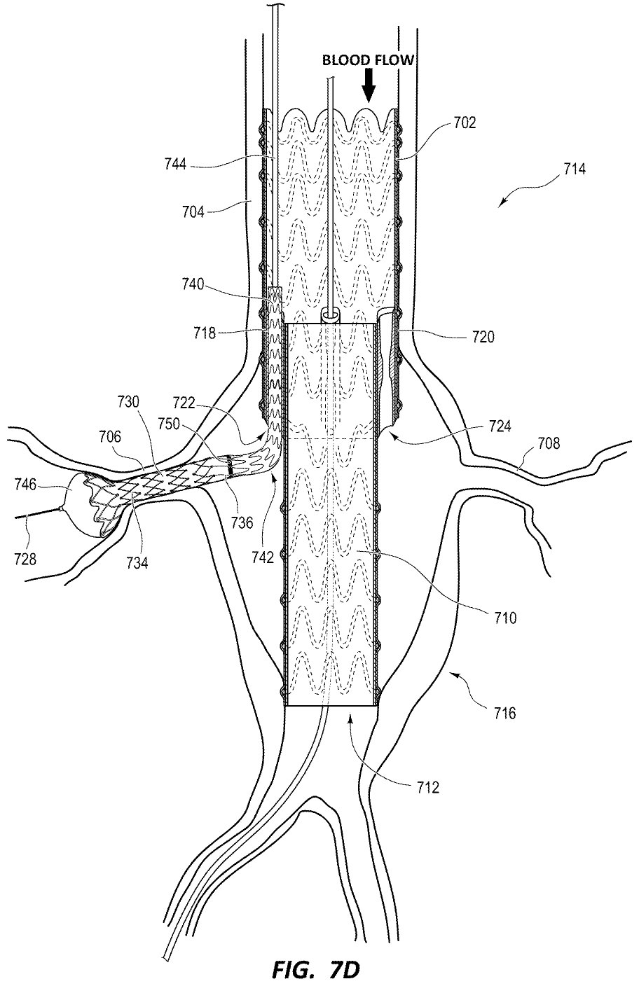

In some cases, a hybrid stent prosthesis 730 is used in conjunction with another prosthesis. FIG. 7A through FIG. 7D illustrate together the use of a fenestrated endovascular prosthesis 702 with a hybrid stent prosthesis 730, according to embodiments herein.

As illustrated in FIG. 7A through FIG. 7D, the fenestrated endovascular prosthesis 702 may be used to bypass a damaged section of the aorta 704, such as a section of the aorta 704 having an aneurysm 750 at or near the locations of a first renal artery 706 and a second renal artery 708 extending from the aorta 704, as illustrated. When in the illustrated deployed configuration, the fenestrated endovascular prosthesis 702 may include a tubular body 710 having a bore 712. In the deployed configuration, each of a proximal portion 714 and a distal portion 716 of the fenestrated endovascular prosthesis 702 couples with healthy arterial tissue of the aorta 704.

In the illustrated example, the fenestrated endovascular prosthesis 702 is used to correct the aneurysm 750 of the aorta 704 by providing a more desirable parallel structure for blood flow down through the aorta 704 in the distal direction. Further, in order to provide necessary flow to (at least) each of the first renal artery 706 and the second renal artery 708, the fenestrated endovascular prosthesis 702 is outfitted with (at least) a first fenestration tube 718 (corresponding to the first renal artery 706) and the second fenestration tube 720 (corresponding to the second renal artery 708). Each of the first fenestration tube 718 and the second fenestration tube 720 may include an opening at a proximal end that is in fluid communication with the bore 712 of the fenestrated endovascular prosthesis 702 and an opening at a distal end that is in fluid communication with the zone exterior to the fenestrated endovascular prosthesis 702. Accordingly, the first fenestration tube 718 may be understood to include a first lumen 722 in fluid communication with the bore 712 and the second fenestration tube 720 may be understood to include a second lumen 724 in fluid communication with the bore 712.

Note that the fenestration tubes 718, 720 may be defined by layers of the tubular body 710, or by separate materials. The fenestrated endovascular prosthesis 702 can be deployed with the fenestration tubes 718, 720 initially in a sealed configuration such that blood flow through the fenestration tubes 718, 720 to the exterior of the fenestrated endovascular prosthesis 702 is prevented.

Embodiments for a method for deploying a hybrid stent prosthesis 730 within the first lumen 722 of the first fenestration tube 718 of the fenestrated endovascular prosthesis 702 and within the first renal artery 706 after the fenestrated endovascular prosthesis 702 has been deployed are now described. In such cases, the hybrid stent prosthesis 730 may be deployed in the first fenestration tube 718 and the first renal artery 706 such that it provides a path for blood to flow between the bore 712 of the fenestrated endovascular prosthesis 702 and the first renal artery 706 (e.g., that is improved over the pathing that would occur through the aneurysm 750 without such a structured path).

It may be understood in such circumstances that the fenestrated endovascular prosthesis 702 is a “parent prosthesis” to the hybrid stent prosthesis 730, where a parent prosthesis is a prosthesis that receives part of a hybrid stent prosthesis as part of the deployment of the hybrid stent prosthesis (as will be shown). It may also be understood in such circumstances that the “deployment site” for the hybrid stent prosthesis 730 is the region between and/or at the first fenestration tube 718 and the first renal artery 706.

A guidewire 728 is provided that runs from an insertion site at the proximal end of the patient, down through the fenestrated endovascular prosthesis 702, out the first fenestration tube 718, and into the first renal artery 706, in the manner illustrated. During the placement of the guidewire 728 in this position indicated, the guidewire 728 may be used to un-seal the first fenestration tube 718 from a sealed configuration to an open configuration.

A distal end of the delivery catheter system 726 may incorporate a deployment system for the hybrid stent prosthesis 730, as such deployment systems are described herein. The guidewire 728 may be run inside a lumen of an elongate tubular member of the delivery catheter system 726, such that the delivery catheter system 726 accordingly follows the guidewire 728 as the delivery catheter system 726 enters the body of the patient and is advanced in the distal direction, as illustrated. The elongate tubular member so used may also serve as the elongate tubular member of the deployment system of the delivery catheter system 726 as is described herein. The delivery catheter system 726 may include a catheter sleeve 732 that may also serve as the catheter sleeve of the deployment system of the delivery catheter system 726 in as is described herein.

The delivery catheter system 726 may be advanced distally through the patient in the manner shown until the distal end of the delivery catheter system 726 (which includes the deployment system for the hybrid stent prosthesis 730) is located at the deployment site, which in the illustrated case is the region between and/or at the first fenestration tube 718 and the first renal artery 706.

FIG. 7B illustrates that once the deployment site is reached, the catheter sleeve 732 may be retracted away from the balloon-expandable stent frame 734 of the hybrid stent prosthesis 730. This may occur in response to an actuation of one or more elements of the delivery catheter system 726 by a practitioner (e.g., as found on a handle of the delivery catheter system 726 (not illustrated) that remains on the outside of the body of the patient). This may be done such that the balloon-expandable stent frame 734 of the hybrid stent prosthesis 730 is sited at the first renal artery 706, as illustrated.

Note that FIG. 7B illustrates a case where the hybrid stent prosthesis 730 includes a transition region 736, and where the catheter sleeve 732 has been retracted such that the transition region 736 is (also) exposed. The practitioner may under imaging, reference the marker 752 to understand when the catheter sleeve 732 has been retracted to a desired position that frees the balloon-expandable stent frame 734 and the transition region 736 but that retains the self-expanding stent frame 740 in the constrained position (e.g., as illustrated in FIG. 7B).

Once the catheter sleeve 732 is retracted away from the balloon-expandable stent frame 734, a first balloon 738 of the deployment system of the delivery catheter system 726 may be inflated. To inflate the first balloon 738, a practitioner may actuate a control for this behavior on a handle of the delivery catheter system 726 that remains on the outside of the body of the patient. This actuation may cause fluid (e.g., air, saline, etc.) to flow through one or more lumens of the delivery catheter system 726/the deployment system of the delivery catheter system 726 and into the first balloon 738 in order to inflate the first balloon 738, as such lumens are described herein (e.g., the lumens 628 of FIG. 6).

The inflation of the first balloon 738 expands the balloon-expandable stent frame 734 out from the collapsed configuration to a deployed configuration, which causes at least a portion of the outside surface of the hybrid stent prosthesis 730 corresponding to the balloon-expandable stent frame 734 to come into contact with the walls of the first renal artery 706 (as illustrated in FIG. 7B). This contact may act to seat or anchor the portion of the hybrid stent prosthesis 730 corresponding to the balloon-expandable stent frame 734 in the desired location within the first renal artery 706.

Once the first balloon 738 has caused the deployment of the portion of the hybrid stent prosthesis 730 corresponding to the balloon-expandable stent frame 734, it may be deflated. To deflate the first balloon 738, a practitioner may actuate a control for this behavior on a handle of the delivery catheter system 726 that remains on the outside of the body of the patient. This actuation may cause fluid to retract out of the first balloon 738 and back through one or more lumens of the delivery catheter system 726/the deployment system of the delivery catheter system 726 in order to deflate the first balloon 738.

As illustrated in FIG. 7C, with the portion of the hybrid stent prosthesis 730 corresponding to the balloon-expandable stent frame 734 in a deployed configuration that acts to seat or anchor the hybrid stent prosthesis 730, the catheter sleeve 732 may be further retracted, such that it retracts away from the self-expanding stent frame 740 of the hybrid stent prosthesis 730. Accordingly, because the self-expanding stent frame 740 is no longer constrained by the catheter sleeve 732, the self-expanding stent frame 740 automatically expands into a deployed configuration.

The expansion of the self-expanding stent frame 740 causes at least a portion of the outside surface of the hybrid stent prosthesis 730 corresponding to the self-expanding stent frame 740 to come into contact with the walls of the first lumen 722 of the first fenestration tube 718 (as illustrated in FIG. 7C). This contact may act to seat or anchor the portion of the hybrid stent prosthesis 730 corresponding to the self-expanding stent frame 740 in the desired location within the first fenestration tube 718 of the fenestrated endovascular prosthesis 702.

Note that the positioning and constituent makeup of the hybrid stent prosthesis 730 is such that when the hybrid stent prosthesis 730 is in a deployed configuration at the deployment site, the portion of the hybrid stent prosthesis 730 that runs through a turn region 742 corresponds to the self-expanding stent frame 740 (e.g., rather than the transition region 736 and/or the balloon-expandable stent frame 734). This may be purposefully arranged in recognition that the self-expanding stent frame 740 may have particular qualities that act to preserve the desired shape of the hybrid stent prosthesis 730 even when it is deflected in order to follow the illustrated route through the turn region 742. For example, the self-expanding stent frame 740 may be configured to tend to keep to a desirable lumen diameter and/or desirable lumen shape for the portion of the self-expanding stent frame 740 that is deflected by an amount necessary to run through the turn region 742, as illustrated in FIG. 7C.

Once the expansion of the self-expanding stent frame 740 is complete, the delivery catheter system 726 may be retracted out of the body of the patient entirely (leaving the guidewire 728 in place).

As can be seen in FIG. 7C, at this stage of deployment, due to the bell shape of the first renal artery 706, it may be that a distal end of the hybrid stent prosthesis 730 does not fully follow or track the walls of the first renal artery 706. This may result in the existence of voids 748 corresponding to the open space between the outside surface of the distal end of the hybrid stent prosthesis 730 and the walls of the first renal artery 706 in the locations where the diameter of the first renal artery 706 “bells” larger. These voids 748 may be undesirable due to blood pooling that may occur therein (which may lead to adverse effects such as blood clotting).

In order to remove the voids 748, a second/additional expansion of the distal end of the hybrid stent prosthesis 730 may be performed. A secondary inflation device 744 may be sent along the guidewire 728 to deliver a second balloon 746 to the distal end of the hybrid stent prosthesis 730 (which, as discussed above, corresponds to the balloon-expandable stent frame 734 of the hybrid stent prosthesis 730). The secondary inflation device 744 may be positioned such that when the second balloon 746 found on the secondary inflation device 744 is inflated, it engages with the distal end of the hybrid stent prosthesis 730. Analogously to the first balloon 738, the second balloon 746 may be inflatable/deflatable by a practitioner using the secondary inflation device 744 by actuating a control on a handle of the secondary inflation device 744 that remains outside the body of the patient and that controls the flow of a fluid through a lumen of the secondary inflation device 744 that is in fluid communication with the second balloon 746.

Then, when the second balloon 746 is inflated, it may cause the portion of the balloon-expandable stent frame 734 corresponding to the distal end of the hybrid stent prosthesis 730 to (further) expand to run along the walls of the first renal artery 706. As can be seen in FIG. 7D, the second balloon 746 may be generally bell shaped in correspondence to the outward belling of the first renal artery 706 (but this is not strictly required). It is noted that the particular shape for the second balloon 746 shown in FIG. 7D is given by way of example and not by way of limitation.

In some embodiments, it may be that, prior to inflation, the secondary inflation device 744 is advanced such that the second balloon 746 is at a location that is past the distal end of the hybrid stent prosthesis 730. Then, the second balloon 746 may be inflated and then pulled back into the distal end of the hybrid stent prosthesis 730 to cause the corresponding portion of the balloon-expandable stent frame 734 to (further) expand as desired.

Whatever the case may be, once inflated, the second balloon 746 may be motivated within the first renal artery 706/the distal end of the hybrid stent prosthesis 730 (e.g., by a practitioner using the secondary inflation device 744 to push and/or pull and/or adjust the inflation of the second balloon 746) to adjust the particular characteristics of the (further) expansion of the distal end of the hybrid stent prosthesis 730.

It is contemplated that, if deemed useful to achieve a desired expansion of the distal end of the hybrid stent prosthesis 730, the second balloon 746 may be deflated, the secondary inflation device 744 may be retracted out of the body of the patient (leaving the guidewire 728), and an additional inflation device having another balloon may be advanced along the guidewire 728 and used in a manner similar to that which has been described in relation to the secondary inflation device 744. Any such additional balloon may be, for example, of a different shape than the second balloon 746 (thereby allowing for separate shaping possibilities from the second balloon 746). This process for the use of an additional balloon may be repeated as many times as is deemed desirable to achieve a desired expansion of the distal end of the hybrid stent prosthesis 730.

Generally speaking, shapes for a balloon for performing the further expansion of the distal end of the hybrid stent prosthesis 730 as has been described may vary. For example, a balloon with a smooth bell shape may be used. In other cases, a bell-shaped stepped balloon may be used (e.g., to give the practitioner a multiple stepped-surface flexibility for adjusting the expansion of the distal end of the hybrid stent prosthesis 730). It is further contemplated that, a balloon that is not generally bell shaped may be used in some circumstances.

Note that while FIG. 7A through FIG. 7D provide description and related illustrations for the deployment of the hybrid stent prosthesis 730 within the first lumen 722 of the first fenestration tube 718 of the fenestrated endovascular prosthesis 702 and within the first renal artery 706, it will be understood that an analogous, second process using a second hybrid stent prosthesis within the second lumen 724 of the second fenestration tube 720 of the fenestrated endovascular prosthesis 702 and within the second renal artery 708 may additionally be performed.

Additionally, note that the description in FIG. 7A through FIG. 7D regarding the deployment of the hybrid stent prosthesis 730 into a renal artery (the first renal artery 706) is given by way of example and not by way of limitation. It is contemplated that arteries other than a renal artery may be targeted for use with a hybrid stent prosthesis, with processes used in such cases being analogous to those described in relation to the deployment of the hybrid stent prosthesis 730 into the first renal artery 706 in relation to FIG. 7A through FIG. 7D. For example, in some cases, it may be that a hybrid stent prosthesis may be deployed instead into, for example, a mesenteric artery (and, e.g., a fenestrated endovascular prosthesis in the aorta) in such an analogous fashion.

Note also that while FIG. 7A through FIG. 7D illustrate the case of a deployment of the hybrid stent prosthesis 730 at a deployment site within the aorta 704, this is only one possible example. It is contemplated that a hybrid stent prosthesis as described herein may be utilized in other locations within the anatomy of a patient, as the case may be. Relatedly, it is also contemplated that a hybrid stent prosthesis as described herein may be deployed with/into a different parent prosthesis (other than the fenestrated endovascular prosthesis 702) that is configured for use at a deployment site that is other than the illustrated location within the aorta 704, as the case may be.

Note further that while the use of a parent prosthesis with the hybrid stent prosthesis is given in examples herein, the use/existence of a parent prosthesis is not strictly required in order for the hybrid stent prosthesis itself to be used/deployed within the anatomy of a patient in at least some circumstances.

Finally, note that while FIG. 7A through FIG. 7D illustrate that the portion of the hybrid stent prosthesis 730 that corresponds to the self-expanding stent frame 740 is deployed proximally and the portion of the hybrid stent prosthesis 730 that corresponds to the balloon-expandable stent frame 734 is deployed distally, this is given by way of one example and not by way of limitation. For example, in some deployment scenarios for a hybrid stent prosthesis, it may be that the portion of a hybrid stent prosthesis corresponding to a balloon-expandable stent frame is deployed proximally, while the portion of that hybrid stent prosthesis corresponding to a self-expanding stent frame is deployed distally. This may be the case with or without the application of such a hybrid stent prosthesis in/with a parent prosthesis.

In one exemplary case, a proximal placement of a portion of a hybrid stent prosthesis that corresponds to a balloon-expandable stent frame may be useful in a “snorkel” situation in which a distal portion of the hybrid stent prosthesis runs along narrower anatomy while a proximal portion of the hybrid stent prosthesis runs parallel to a larger trunk. In such cases, it may be desirable for proximal portion of the hybrid stent prosthesis to have a high radial strength corresponding to the balloon-expandable stent frame.

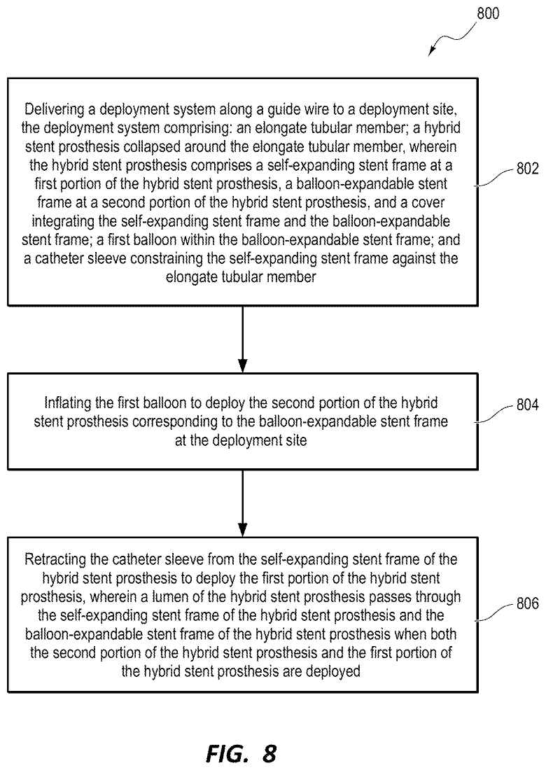

FIG. 8 illustrates a method 800 of deploying a hybrid stent prosthesis, according to embodiments herein. The method 800 includes delivering 802 a deployment system along a guide wire to a deployment site, the deployment system comprising: an elongate tubular member; the hybrid stent prosthesis collapsed around the elongate tubular member, wherein the hybrid stent prosthesis comprises a self-expanding stent frame at a first portion of the hybrid stent prosthesis, a balloon-expandable stent frame at a second portion of the hybrid stent prosthesis, and a cover integrating the self-expanding stent frame and the balloon-expandable stent frame; a first balloon within the balloon-expandable stent frame; and a catheter sleeve constraining the self-expanding stent frame against the elongate tubular member.

The method 800 further includes inflating 804 the first balloon to deploy the second portion of the hybrid stent prosthesis corresponding to the balloon-expandable stent frame at the deployment site.

The method 800 further includes retracting 806 the catheter sleeve from the self-expanding stent frame of the hybrid stent prosthesis to deploy the first portion of the hybrid stent prosthesis, wherein a lumen of the hybrid stent prosthesis passes through the self-expanding stent frame of the hybrid stent prosthesis and the balloon-expandable stent frame of the hybrid stent prosthesis when both the second portion of the hybrid stent prosthesis and the first portion of the hybrid stent prosthesis are deployed.

In some embodiments, inflating 804 the first balloon to deploy the second portion of the hybrid stent prosthesis is performed before retracting 806 the catheter sleeve from the self-expanding stent frame of the hybrid stent prosthesis to deploy the first portion of the hybrid stent prosthesis. In some embodiments, retracting 806 the catheter sleeve from the self-expanding stent frame of the hybrid stent prosthesis to deploy the first portion of the hybrid stent prosthesis is performed before inflating 804 the first balloon to deploy the second portion of the hybrid stent prosthesis.

In some embodiments, the method 800 further includes delivering a second balloon to the deployment site along the guide wire and using the second balloon to adjust the second portion of the hybrid stent prosthesis corresponding to the balloon-expandable stent frame.

In some embodiments of the method 800, the deployment system is delivered along the guide wire to the deployment site through a parent prosthesis.

In some embodiments of the method 800, the second portion of the hybrid stent prosthesis is deployed in a renal artery at the deployment site, and the first portion of the hybrid stent prosthesis is deployed within a lumen of a parent prosthesis at the deployment site.

In some embodiments of the method 800, the first portion of the hybrid stent prosthesis is deployed proximally and the second portion of the hybrid stent prosthesis is deployed distally.

In some embodiments of the method 800, the second portion of the hybrid stent prosthesis is deployed in a mesenteric artery at the deployment site, and wherein a first portion of the hybrid stent prosthesis is deployed within a lumen of a parent prosthesis at the deployment site.

In some embodiments of the method 800, the cover further comprises a transition zone between the self-expanding stent frame and the balloon-expandable stent frame.

In some embodiments of the method 800, when the hybrid stent prosthesis is in a collapsed configuration, the catheter sleeve further covers the balloon-expandable stent frame, and the method 800 further includes retracting the catheter sleeve from the balloon-expandable stent frame prior to inflating the first balloon.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A hybrid stent prosthesis comprising:
a self-expanding stent frame;
a balloon-expandable stent frame; and
a cover integrating the self-expanding stent frame and the balloon-expandable stent frame such that when the hybrid stent prosthesis is in a deployed configuration, a lumen of the hybrid stent prosthesis passes through the self-expanding stent frame and the balloon-expandable stent frame.

2. The hybrid stent prosthesis of claim 1, wherein a luminal surface of the cover is continuous between the self-expanding stent frame and the balloon expandable stent frame.

3. The hybrid stent prosthesis of claim 1, wherein the cover further comprises a transition zone between the self-expanding stent frame and the balloon-expandable stent frame.

4. The hybrid stent prosthesis of claim 3, wherein the transition zone comprises a longitudinal beading extending from the self-expanding stent frame to the balloon-expandable stent frame.

5. The hybrid stent prosthesis of claim 3, wherein the transition zone comprises a helical beading extending from the self-expanding stent frame to the balloon-expandable stent frame.

6. The hybrid stent prosthesis of claim 3, wherein a first layering of the cover in the transition zone is different than a second layering of the cover at an end of one of the self-expanding stent frame and the balloon-expandable stent frame that is adjacent to the transition zone.

7. The hybrid stent prosthesis of claim 3, wherein the transition zone of the cover and regions of the cover integrating the self-expanding stent frame and the balloon-expandable stent frame include a single, continuous material.

8. The hybrid stent prosthesis of claim 1, wherein a first nominal diameter of an end portion of the cover that corresponds to the balloon-expandable stent frame is larger than a second nominal diameter of the balloon-expandable stent frame.

9. The hybrid stent prosthesis of claim 1, wherein an end portion of the cover corresponding to the balloon-expandable stent frame has an elasticity that functionally corresponds to a maximum deployable diameter of the balloon-expandable stent frame.

10. The hybrid stent prosthesis of claim 1, further comprising an additional self-expanding stent frame, wherein the cover integrates the additional self-expanding stent frame with the balloon-expandable stent frame positioned between the self-expanding stent frame and the additional self-expanding stent frame.

11. The hybrid stent prosthesis of claim 1, further comprising an additional balloon-expandable stent frame, wherein the cover integrates the additional balloon-expandable stent frame with the self-expanding stent frame positioned between the balloon-expandable stent frame and the additional balloon-expandable stent frame.

12. A deployment system, comprising:
an elongate tubular member;
a hybrid stent prosthesis collapsed around the elongate tubular member comprising:
a self-expanding stent frame;
a balloon-expandable stent frame; and
a cover integrating the self-expanding stent frame and the balloon-expandable stent frame such that when the hybrid stent prosthesis is in a deployed configuration, a lumen of the hybrid stent prosthesis passes through the self-expanding stent frame and the balloon-expandable stent frame;
a balloon within the balloon-expandable stent frame that is inflatable through the elongate tubular member to deploy a portion of the hybrid stent prosthesis corresponding to the balloon-expandable stent frame; and
a catheter sleeve constraining the self-expanding stent frame of the hybrid stent prosthesis against the elongate tubular member.

13. The deployment system of claim 12, wherein the catheter sleeve further covers the balloon-expandable stent frame of the hybrid stent prosthesis.

14. A method of deploying a hybrid stent prosthesis, comprising:
delivering a deployment system along a guide wire to a deployment site, the deployment system comprising:
an elongate tubular member;
the hybrid stent prosthesis collapsed around the elongate tubular member, wherein the hybrid stent prosthesis comprises:
a self-expanding stent frame at a first portion of the hybrid stent prosthesis;
a balloon-expandable stent frame at a second portion of the hybrid stent prosthesis; and
a cover integrating the self-expanding stent frame and the balloon-expandable stent frame;
a first balloon within the balloon-expandable stent frame; and
a catheter sleeve constraining the self-expanding stent frame against the elongate tubular member; and
deploying the first portion and the second portion of the hybrid stent prosthesis,
wherein a lumen of the hybrid stent prosthesis passes through the self-expanding stent frame of the hybrid stent prosthesis and the balloon-expandable stent frame of the hybrid stent prosthesis when both the second portion of the hybrid stent prosthesis and the first portion of the hybrid stent prosthesis are deployed.

15. The method of claim 14, wherein deploying the first portion and the second portion of the hybrid stent prosthesis comprises:
inflating the first balloon to deploy the second portion of the hybrid stent prosthesis corresponding to the balloon-expandable stent frame at the deployment site; and
after inflating the first balloon to deploy the second portion of the hybrid stent prosthesis, retracting the catheter sleeve from the self-expanding stent frame of the hybrid stent prosthesis to deploy the first portion of the hybrid stent prosthesis.

16. The method of claim 14, wherein deploying the first portion and the second portion of the hybrid stent prosthesis comprises:
retracting the catheter sleeve from the self-expanding stent frame of the hybrid stent prosthesis to deploy the first portion of the hybrid stent prosthesis; and
after retracting the catheter sleeve from the self-expanding stent frame of the hybrid stent prosthesis, inflating the first balloon to deploy the second portion of the hybrid stent prosthesis corresponding to the balloon-expandable stent frame at the deployment site.

17. The method of claim 14, further comprising:
delivering a second balloon to the deployment site along the guide wire; and
using the second balloon to adjust the second portion of the hybrid stent prosthesis corresponding to the balloon-expandable stent frame.

18. The method of claim 14, wherein the deployment system is delivered along the guide wire to the deployment site through a parent prosthesis.

19. The method of claim 14, wherein the second portion of the hybrid stent prosthesis is deployed in a renal artery at the deployment site, and wherein the first portion of the hybrid stent prosthesis is deployed within a lumen of a parent prosthesis at the deployment site.

20. The method of claim 14, wherein the first portion of the hybrid stent prosthesis is deployed proximally and the second portion of the hybrid stent prosthesis is deployed distally.
